(19) United States
(12) Patent Application Publication
(10) Pub. No.: US 2026/0078294 A1
(43) Pub. Date: Mar. 19, 2026

(54) ENZYMES FOR CONTROLLING MICROBIOLOGICALLY INFLUENCED CORROSION

(71) Applicant: LANXESS Corporation, Pittsburgh, PA (US)

(72) Inventors: Huiqing Zheng, Glen Mills, PA (US); Henry Ophardt, Wilmington, DE (US); Angela Iglesias, Romansville, PA (US); Nora Eibergen, North Aurora, IL (US)

(21) Appl. No.: 19/107,063

(22) PCT Filed: Aug. 30, 2023
(86) PCT No.: PCT/US2023/073141

§ 371 Date: Feb. 27, 2025

Related U.S. Application Data
(60) Provisional application No. 63/403,083, filed on Sep. 1, 2022.

(51) Int. Cl. C09K 8/524; C09K 8/54; C12N 9/24; C12N 9/26; C12N 9/42; C12N 9/50; C23F 15/00

(52) U.S. Cl. CPC C09K 8/524; C09K 8/54; C12N 9/2411; C12N 9/2437; C12N 9/2445; C12N 9/248; C12N 9/50; C23F 15/00

(57) ABSTRACT

Disclosed herein are methods and compositions for controlling microbiologically influenced corrosion (MIC) through the use of certain enzymes. The methods and compositions are useful for applications susceptible to or in need of treatment or remediation for MIC associated with biofilms formed on metal surfaces under anaerobic conditions, such as in equipment or structures within oil and gas systems, particularly the production, transportation and storage infrastructure and equipment for oil and gas applications.

### FIELD OF INVENTION

This invention relates to an enzyme-based solution for controlling microbiologically influenced corrosion (MIC), such as in oil and gas applications.

### BACKGROUND OF THE INVENTION

Microbiologically influenced corrosion (MIC) is the terminology commonly applied where the actions of microorganisms influence the corrosion process (Skovhus et al., Journal of Biotechnology, 256 (2017) 31-45). MIC is frequently observed at oil production sites and in transport equipment, among other types of equipment involved in the oil and gas production, transportation and storage industries. MIC poses severe operational, environmental, and safety problems to these industries, particularly with respect to corrosion of equipment used in the storage, processing, and/or transport of oil and gas crude and/or refined products. Costs resulting from MIC in these industries due to repair and replacement of damaged equipment, spoiled oil, environmental clean-up, and injury-related health care, are estimated to amount to hundreds of millions to over several billion USD per year.

Biofilms that form on the surfaces of metal components are thought to be the primary causative agent triggering or facilitating such corrosion, particularly biofilms formed in anaerobic environments involving anaerobic microorganisms associated with the production of harmful gases (e.g., hydrogen sulfide) and other agents or byproducts which are highly corrosive or create corrosive conditions, in addition to directly affecting metallic integrity. Hydrogen sulfide also poses health and safety concerns to workers in the industry.

Sulfate-reducing microorganisms (SRM) are commonly found in the produced water of oil and gas systems. SRM naturally utilize sulfate as the terminal electron acceptor during anaerobic respiration through dissimilatory sulfate reduction, resulting in corrosive hydrogen sulfide (H₂S) production as a metabolic by-product. H₂S is a toxic gas which can sour oil reservoirs resulting in devalued hydrocarbons as well as health, safety and environmental issues topside. Furthermore, when SRM attach on metal surfaces, they can cause microbiologically influenced corrosion by directly taking electrons from the metals and/or secreting H₂S. Together, MIC and souring result in substantial economic losses and are challenging to remediate in the oil and gas industry.

Common corrosion inhibitors are composed of amines, condensation products of fatty acids with polyamines, imidazolines, and/or quaternary ammonium compounds. Among the most frequently used corrosion inhibitors in crude oil and natural gas extraction are imidazoline derivatives and benzyldimethylalkylammonium chlorides.

U.S. Pat. No. 5,789,239A discloses the use of certain enzymes in combination with a short-chained glycol component for the avoidance of slime formation and/or for the removal of biofilm on surfaces of water-bearing system. U.S. Pat. No. 5,411,666A discloses compositions for removing biofilm and controlling its development in industrial water systems comprising at least two biologically produced enzymes and a surface active agent, preferably an anionic surfactant. These references do not relate to or address MIC associated with biofilms formed under anaerobic conditions, which create particularly challenging corrosive environments for remediating MIC, especially in the oil and gas production, transportation and storage industries.

EP2599849A1 discloses a method of inhibiting corrosion of a downhole casing comprising contacting the inner surface of the casing with an oxidoreductase enzyme, especially peroxidase enzymes. No examples or data are disclosed with respect to using an oxidoreductase enzyme to reduce MIC. As described in the Examples of the present disclosure, it was found that peroxidase provided no reduction in MIC associated with biofilms formed under anaerobic conditions.

U.S. Pat. No. 10,017,403B2 discloses methods of treating drilling fluids, frac fluids, flowback water and disposal water using peracetic acid/hydrogen peroxide and peroxide-reducing enzymes.

U.S. Pat. No. 9,732,369B2 discloses that a variety of strategies have been developed or discussed to mitigate the corrosive effects of MIC and/or the biofilms that contribute to or cause MIC such as the use of corrosion-resistant metals, temperature control, pH control, radiation, filtration, protective coatings, the use of corrosion inhibitors or other chemical controls (e.g., biocides, oxidizers, acids, alkalis), bacteriological controls (e.g., phages, enzymes, parasitic bacteria, antibodies, competitive microflora), pigging (i.e., mechanical delamination of corrosion products), anodic and cathodic protection, and modulation of nutrient levels. More specifically, the reference discloses a combination treatment of biocide application and pigging.

Traditionally, souring is mitigated by applying H₂S scavengers in production fluids. However, this strategy does not solve MIC issues caused by direct electron uptake by SRM. Biocides are an alternative to control the microbial population in the oil and gas field for souring and MIC control. However, biocides are often less effective in killing sessile cells imbedded in the biofilm as they may be unable to adequately penetrate or permeate the extracellular matrix of the biofilm.

Overall, while mitigation techniques to reduce MIC are available, limitations exist to their effectiveness and/or they are not practical in the industry due to high cost.

Therefore, there is an urgent need to develop a novel solution to remediate MIC associated with biofilms formed under anaerobic conditions, particularly in the oil and gas production, transportation and storage industries.

### SUMMARY OF THE INVENTION

In one aspect, the present disclosure includes a method of controlling microbiologically influenced corrosion (MIC) of a site of interest, comprising contacting a biofilm formed under anaerobic conditions on a metal surface of the site of interest with an effective amount of an enzyme selected from the group consisting of a protease, a carbohydrolase, a deoxyribonuclease (DNase) and a combination thereof.

In another aspect, the present disclosure includes a composition (e.g., a treatment fluid) comprising an enzyme selected from the group consisting of a protease, a carbohydrolase, a DNase and a combination thereof in an amount effective to control microbiologically influenced corrosion (MIC).

The methods and compositions disclosed herein are useful for applications susceptible to or in need of treatment or remediation for MIC associated with biofilms formed on metal surfaces under anaerobic conditions. Particular exemplary sites of interest applicable to the present invention include equipment or structures within oil and gas systems, particularly the production, transportation and storage infrastructure and equipment for oil and gas (e.g., in rigs, storage and base structures, pipelines, tanks, etc.).

### DETAILED DESCRIPTION OF THE INVENTION

Unless otherwise specified, the following terms as used in the present disclosure have the meanings defined below:

The term “microbiologically influenced corrosion,” “MIC” and the like refer to corrosion influenced directly or indirectly by the effects of microorganisms (including the products of their metabolic activity) in biofilms formed under anaerobic conditions on metal surfaces. “Microbiologically influenced corrosion” and “microbially induced corrosion” are used interchangeably herein.

The term “biofilm” refers to a multicellular bacterial community composed of surface-associated microbial cells that are held together by a self-developed matrix of extracellular polymeric substance. The biofilms relevant to the present disclosure are formed under anaerobic conditions.

The term “effective amount” and the like mean an amount or concentration of the presently disclosed enzymes, the amount or concentration being effective for controlling MIC.

The term “controlling microbiologically influenced corrosion (MIC)” and the like of a site of interest refer to the ability of the enzymes of the present disclosure to reduce or inhibit MIC as compared to the level of MIC in the absence of treatment. For example, controlling MIC may be evaluated through weight loss or corrosion rate metal coupon testing (e.g., according to the protocol NACE Standard RP0775-2005) in the field using metal coupons positioned at a site of interest in an anaerobic environment or by simulation using metal coupons that have been subjected to an inoculum of microorganisms to form biofilms under anaerobic conditions, such as biofilms comprising SRM, (e.g., using a flow loop or other means to expose the metal coupons to an inoculum for a time period, such as for two or more weeks). The control of MIC may be determined by comparing the measured weight loss or the corrosion rate of the metal coupon(s) contacted with the enzyme(s) of the present disclosure (treated metal coupon) to the measured weight loss or corrosion rate of the untreated control coupon(s) and may be expressed as the percentage weight loss reduction or the percentage corrosion rate reduction relative to the untreated control. If the comparison is made on the basis of measured weight loss, the weight loss should be determined over an equivalent period of time for the untreated and treated metal coupons. If the comparison is made on the basis of corrosion rate, the corrosion rate is preferably determined over an equivalent period of time for the untreated and treated metal coupons. Preferably, the enzymes of the present disclosure are applied at an amount or concentration and for a contact time (and at intervals or frequencies, if desired) to provide a weight loss reduction (% relative to untreated control) of at least 10%, preferably such as at least 20%, such as at least 30%, or such as at least 40%. For example, as demonstrated in the Examples section herein, a weight loss reduction of at least 20%, at least 30%, or at least 40% was achieved after three or four weekly treatments (once-per-week treatment). Preferably, the enzymes of the present disclosure are applied at an amount or concentration and for a contact time (and at intervals or frequencies, if desired) to provide a corrosion rate reduction of at least 10%, preferably such as at least 20%, such as at least 30%, or such as at least 40%. For example, as demonstrated in the Examples section herein, a corrosion rate reduction of at least 20%, or at least 30%, or at least 40% was achieved after three or four weekly treatments (once-per-week treatment).

The term “protease” or “proteinase” refers to enzymes that catalyze the hydrolysis of peptide bonds.

The term “metalloprotease” refers to a protease having one or more metal ions in the binding/active site.

The term “serine protease” refers to a protease in which there is an essential serine residue at the active site.

The term “thermostable protease” refers to a protease that is heat stable, as is known in the art. Preferably, the thermostable protease is stable above 40° C., such as above 50° C., such as above 60° C., such as above 70° C., such as above 80° C., such as above 90° C., such as above 100° C., such as above 110° C., such as above 120° C. or higher. Thermostability may often fall within a range from about 45° C. to about 120° C.

The term “alkaline protease” refers to a protease that is active in a neutral to alkaline pH range.

The term “carbohydrolase” refers to enzymes that catalyze the hydrolysis of carbohydrates, preferably the hydrolysis of glycosidic bonds.

The term “deoxyribonuclease (DNase)” refers to an enzyme that catalyzes the hydrolytic cleavage of phosphodiester linkages in DNA.

Disclosed is a method of controlling MIC of a site of interest, comprising contacting a biofilm formed on a metal surface of the site of interest with an effective amount of an enzyme selected from the group consisting of a protease, a carbohydrolase, a DNase and a combination thereof. The site of interest may be equipment, such as pipelines, tanks, drums, containers, vessels, associated componentry and units and so forth, or structures, such as storage or base structures.

Also disclosed is a composition, such as a treatment fluid, comprising an enzyme selected from the group consisting of a protease, a carbohydrolase, a DNase and a combination thereof in an amount effective to control MIC.

Biofilms are formed by diverse metabolic microorganisms. The biofilms involved in the methods of the present disclosure are those that form under anaerobic conditions and therefore comprise anaerobic microorganisms (inclusive of facultative anaerobic microorganisms which can function under anaerobic conditions). Typically, all or principally all of the microorganisms in the biofilms involved in the presently disclosed methods are anaerobic microorganisms. In this context, “principally all” of the microorganisms would be understood to mean that the formation and activity of the biofilm, including its corrosive nature, is principally driven by or attributed to anaerobic microorganisms.

In many instances, the anaerobic microorganisms comprise sulfate-reducing microorganisms (SRM). Examples of genera of SRM include Desulfovibrio, Desulfocarbo, Desulfobacterium, Desulfobulbus, Desulfoarculus, Desulfobacter, Desulfococcus, Desulfotomaculum, Desulfosporomusa, Desulfosporosinus, Bilophila, Desulfobaculum, Desulfocurvibacter, Desulfocurvus, Desulfohalovibrio, Desulfolutivibrio, Desulfohalobium, Desulfonatronospira, Desulfonatronovibrio, Desulfothermus, Desulfonauticus, Desulfovermiculus, Desulfohalophilus, Desulfatibacillum, Desulfomonas, Thermodesulfovibrio, Archaeoglobus, Thermocladium, Caldivirga, among others.

The anaerobic microorganisms may include methanogenic archaea, iron-reducing bacteria, thiosulfate-reducing bacteria and other anaerobic microorganisms.

The anaerobic microorganisms according to the present disclosure often comprise strictly anaerobic microorganisms, which can survive, grow and carry out metabolic processes in only anaerobic environments. In some embodiments, the formation and activity of the biofilm is attributed, at least in part, to strictly anaerobic microorganisms. For example, in some embodiments, a majority of the microbial composition of the biofilm is strictly anaerobic. In some embodiments, the formation and activity of the biofilm is principally driven by or attributed to strictly anaerobic microorganisms.

In some embodiments, the formation and activity of the biofilm is attributed, at least in part, to anaerobic Deltaproteobacteria. For example, in some embodiments, a majority of the microbial composition of the biofilm is of the anaerobic Deltaproteobacteria class. In some embodiments, the formation and activity of the biofilm is principally driven by or attributed to anaerobic Deltaproteobacteria.

In some embodiments, the formation and activity of the biofilm is attributed, at least in part, to SRM. For example, in some embodiments, a majority of the microbial composition of the biofilm are SRM. In some embodiments, the formation and activity of the biofilm is principally driven by or attributed to SRM.

Techniques for evaluating the microbial composition are known in the field, such as by PCR/DGGE (Polymerase Chain Reaction/Denaturing Gradient Gel Electrophoresis) and/or NGS (Next Generation Sequencing methodology), as described, for example, in Eibergen et al., “Comparing Oilfield Biocides for Corrosion Control Using a Laboratory Method for MIC Generation Under Oilfield-Relevant Conditions,” SPE International Oilfield Corrosion Conference and Exhibition, SPE-190901-MS (2018) (doi.org/10.2118/190901-MS), which is incorporated herein by reference for this purpose.

In the presently disclosed methods, the MIC of a site of interest may be associated with a corrosion rate. A corrosion rate of a site of interest may be determined, for example, by assessing the corrosion rate of metal coupon(s) positioned at the site of interest (e.g., according to the protocol NACE Standard RP0775-2005). For example, the MIC to be controlled by the methods of the present disclosure may be associated with a corrosion rate of about 1 milli-inch/year (mpy) or higher (i.e., about 0.0254 mm/yr or higher), such as at least 2 mpy (0.0508 mm/yr or higher), such as at least 3 mpy (0.0762 mm/yr or higher), such as at least 4 mpy (0.1016 mm/yr or higher), such as at least 5 mpy (0.127 mm/yr or higher), such as at least 6 mpy (0.1524 mm/yr or higher), such as at least 7 mpy (0.1778 mm/yr or higher) or such as at least 8 mpy (0.2032 mm/yr or higher).

Depending on the environment where the MIC is occurring, very high corrosion rates may be observed (e.g., high localized corrosion), such as a corrosion rate of 10 mpy or higher (0.254 mm/yr or higher), 15 mpy or higher (0.381 mm/yr or higher), 20 mpy or higher (0.508 mm/yr or higher) or higher.

Preferably, the enzymes of the present disclosure are applied at an amount or concentration and for a contact time (and at intervals or frequencies, if desired) to provide a corrosion rate reduction of at least 10%, preferably such as at least 20%, such as at least 30%, or such as at least 40%, compared to the corrosion rate in the absence of treatment.

Controlling MIC of a site of interest using the enzymes of the present disclosure may also or alternatively be evaluated through metal coupon testing (e.g., according to the protocol NACE Standard RP0775-2005) in a simulation setting using metal coupon(s) that have been subjected to an inoculum of microorganisms (e.g., from a consortium relevant to the site of interest) to form biofilms under anaerobic conditions, such as biofilms comprising anaerobic microorganisms as described herein. For example, the metal coupon(s) may be subjected to such an inoculum using a flow loop or other means to expose the metal coupon(s) to the inoculum for a period of time under anaerobic conditions to establish biofilms, such as for one or more, or preferably for two or more weeks. The resulting MIC over time of the untreated metal coupon(s) may be associated with a corrosion rate, such as any of the corrosion rates identified above. In a simulation setting designed to promote the growth of biofilms under anaerobic conditions, the corrosion rate may exceed 25 mpy (0.635 mm/yr), such as 30 mpy or higher (0.762 mm/yr or higher), such as 35 mpy or higher (0.889 mm/yr or higher), such as 40 mpy or higher (1.016 mm/yr or higher), such as 45 mpy or higher (1.143 mm/yr or higher), such as 50 mpy or higher (1.27 mm/yr or higher), such as 55 mpy or higher (1.397 mm/yr or higher), or such as 60 mpy or higher (1.524 mm/yr or higher). Preferably, the enzymes of the present disclosure are applied at an amount or concentration and for a contact time (and at intervals or frequencies, if desired) to provide a corrosion rate reduction of at least 10%, preferably such as at least 20%, such as at least 30%, or such as at least 40%, compared to the corrosion rate in the absence of treatment.

Metal coupons are typically chosen to be representative of the relevant material of the site of interest, such as of the relevant infrastructure or equipment, as described herein.

The methods and compositions of the present disclosure may employ more than one enzyme (i.e., a combination of two or more of the enzymes of the present disclosure).

The contacting step uses an effective amount of the enzyme and occurs for a time sufficient to control MIC. For example, the contact time can range from about 20 minutes to about 12 hours, from about 40 minutes to about 8 hours, from about 1 hour to about 6 hours, from about 1 hour to about 4 hours, or from about 2 hours to about 4 hours, or, in some embodiments, the contact time is about 4 hours. Other contact times are also suitable. The contacting step may also be conducted at intervals or certain frequencies (e.g., daily, weekly, monthly, and so forth or any combination thereof). For example, in many embodiments, the desired control of MIC (e.g., a weight loss or corrosion rate reduction of at least 10%, preferably such as at least 20%, such as at least 30%, or such as at least 40%) is achieved after consecutive weekly treatments, such as once-per-week treatments for two, three, four, or more weeks.

In many embodiments, the site of interest is infrastructure or equipment for refining, storing, or transporting crude or processed oil, such as metal pipelines, transportation or storage containers, refinery processing equipment, etc.

In many other embodiments, the site of interest is infrastructure or equipment for processing, storing, or transporting natural gas, such as metal pipelines, transportation or storage containers, refinery processing equipment, etc.

The compositions of the present disclosure (and those employed in the presently disclosed methods) comprise an effective amount of the enzyme, to which, in accordance with the presently disclosed methods, the biofilms are exposed at the site of interest, such as by introducing the enzyme into affected infrastructure or equipment used for oil and/or gas refining, storing or transportation. For example, a suitable effective amount of the enzyme for contacting the biofilms at the site of interest may be a concentration (mg/L (ppm)) of at least 1 ppm, at least 10 ppm, at least 25 ppm, at least 50 ppm, at least 100 ppm or at least 500 ppm, such as from about 1 ppm to about 500 ppm, from about 5 ppm or about 10 ppm to about 500 ppm, from about 10 ppm to about 250 ppm, or any value or range therebetween. Often, the enzyme concentration is from about 10 ppm to about 150 ppm or from about 25 ppm to about 100 ppm, or about 10 ppm, about 25 ppm, about 50 ppm, about 100 ppm or about 150 ppm. In some embodiments, the enzyme concentration in the composition is about 100 ppm.

The effective amount of the enzyme at the site of interest may also be, for example, at least 0.0001 wt %, at least 0.001 wt %, at least 0.01 wt %, or at least 0.02 wt %, such as from about 0.0001 wt % to about 0.05 wt %, from about 0.001 wt % to about 0.025 wt %, or often from about 0.001 wt % to about 0.02 wt %, based on the total weight of the composition, such as a treatment fluid or the fluid of a pipeline, tank, drum, vessel, container, or other structure or equipment.

Based on the particular application and the site targeted for treatment, the skilled person will be able to determine an appropriate amount of the enzyme to be introduced or delivered to expose the biofilms to an effective amount of the enzyme at the targeted site.

The enzymes are delivered to the site of interest in a medium or carrier for carrying out the contacting step of the present disclosure. Examples of suitable mediums or carriers include, but are not limited to, aqueous-based fluids, such as fresh water, seawater, saltwater, or brine (e.g., water containing or saturated with one or more dissolved salts) and any combination thereof; an aqueous-miscible fluid, such as alcohols, e.g., methanol, ethanol, n-propanol, isopropanol, n-butanol, sec-butanol, isobutanol, and t-butanol; glycerins; glycols, e.g., polyglycols, propylene glycol, and ethylene glycol; polyglycol amines; polyols; any derivative thereof; any in combination with salts, e.g., sodium chloride, calcium chloride, calcium bromide, zinc bromide, potassium carbonate, sodium formate, potassium formate, cesium formate, sodium acetate, potassium acetate, calcium acetate, ammonium acetate, ammonium chloride, ammonium bromide, sodium nitrate, potassium nitrate, ammonium nitrate, ammonium sulfate, calcium nitrate, sodium carbonate, and potassium carbonate. Combinations of one or more aqueous-miscible fluid with an aqueous-based fluid may also serve as carriers.

The medium or carrier may include fluids already present at or surrounding the metal surface of the site of interest, such as in pipelines, tanks and other equipment and structures, e.g., in the production, transportation and storage of oil and gas. For example, the enzymes of the present disclosure may be added to such fluids at or surrounding the metal surface of the site of interest for exposing the biofilm to an effective amount of the enzyme for controlling MIC.

The compositions or treatment fluids of the present disclosure may include one or more additional additives, such as corrosion inhibitors and/or biocides. For example, in some embodiments, the methods of the present disclosure further comprise delivering one or more corrosion inhibitors and/or biocides to the site of interest.

The present application is not limited to any specific technique for contacting the biofilms with the enzymes, and the exact manner of conducting the contacting step will depend on the particular application and the site of interest.

The enzymes of the present disclosure are selected from the group consisting of proteases, carbohydrolases, DNases and combinations thereof. In many embodiments, the enzyme is chosen from a protease, a carbohydrolase and a combination thereof.

In many embodiments, the protease is chosen from a metalloprotease, a thermostable protease, an alkaline protease and a combination thereof.

In many embodiments, the protease is a serine protease.

As understood in the art, thermostable proteases and alkaline proteases are not exclusive categories, that is, proteases can be both thermostable and alkaline proteases. In addition, proteases that are alkaline proteases, thermostable proteases, or alkaline and thermostable proteases can include, but are not limited to, metalloproteases and serine proteases (e.g., alkaline and/or thermostable serine proteases and alkaline and/or thermostable metalloproteases).

The metalloprotease may be of the classification EC 3.4.24, such as thermolysin or thermolysin-like proteases (e.g., from the M4 family of metalloproteases) or of the classification EC 3.4.17, such as carboxypeptidase A, carboxypeptidase B or the like (e.g., from the M14 family of metalloproteases) or other metalloproteases may be used.

The serine protease may be of the classification EC 3.4.21, for example, subtilisin or subtilisin-like proteases (e.g., from the S8 family of serine proteases), trypsin, chymotrypsin or trypsin-like or chymotrypsin-like proteases (e.g., from the S1 family of serine proteases), or other serine proteases, such as serine carboxypeptidases (e.g., of the classification EC 3.4.16).

Thermolysin and thermolysin-like proteases and subtilisin and subtilisin-like proteases are non-limiting examples of thermostable proteases. Non-limiting examples of other thermostable proteases are classified within EC 3.4.17, EC 3.4.21-24, among others.

Subtilisin and subtilisin-like proteases, trypsin and trypsin-like proteases and chymotrypsin and chymotrypsin-like proteases are non-limiting examples of alkaline proteases. Non-limiting examples of other alkaline proteases are classified within EC 3.4.17, EC 3.4.21-24, among others.

In many embodiments, the carbohydrolase is chosen from a glucosidase, an amylase (e.g., alpha-amylase, beta-amylase), a xylanase and a combination thereof. Preferably, the glucosidase is chosen from a cellulase, a glucanase (e.g., beta-glucanase), a beta-glucosidase and a combination thereof.

In many embodiments, the enzyme is selected from the group consisting of a metalloprotease, a serine protease, a thermostable protease, an alkaline protease, a cellulase, a xylanase, an amylase (e.g., alpha-amylase), a glucanase (e.g., beta-glucanase), a beta-glucosidase, and a combination thereof.

In many embodiments, the enzyme is selected from the group consisting of a metalloprotease, a thermostable protease, an alkaline protease, a cellulase, a xylanase, an amylase (e.g., alpha-amylase), a glucanase (e.g., beta-glucanase), a beta-glucosidase, and a combination thereof.

The compositions and methods of the present application may employ one or more than one enzyme of the present disclosure. The enzymes of the present disclosure may be derived from any suitable origin, such as vegetable, animal, bacterial, fungal or yeast origin.

Examples of suitable enzymes are those marketed under the brand names Optimash® DCO+®, Effectenz® P100, PREFERENZ™ P 300, Optimash® AX, Optimash® BG, Optimash® VR, Optimash® F200, Spezyme® HT and combinations thereof.

The methods disclosed herein may also include testing steps (e.g., upstream or downstream) that facilitate knowing whether, how and/or when to administer the enzyme-based treatment. Such additional steps may aim to determine whether a system has a probable MIC risk at a particular site. Other steps may involve subsequent monitoring to evaluate the potential occurrence or extent of MIC, followed by steps to carry out a particular treatment plan comprising the present enzyme-based treatment.

For example, corrosive damage to infrastructure or equipment might be detected as a result of regularly scheduled maintenance. To learn more about the extent and nature of the damage, a user might sample the environmental conditions at various points, e.g., along a pipeline, by assessing properties that would be indicative of biofilm formation in anaerobic environments, such as (a) detection of certain anaerobic bacterial species known to have a role in bacterial corrosion (e.g., sulfate reducing bacteria), (b) detection of certain corrosive metabolites (e.g., presence of organic acids, hydrogen sulfide gas), (c) existence of suitable pH and temperature conditions known to be supportive of biofilm development, (d) presence of an aqueous environment (e.g., extent of water drop-out or separation of a water phase from crude oil), (e) slow flow rate (slower flow rates are conducive to biofilm formation), and (f) existence of high bacterial biomass. The skilled person may also wish to examine physical samples collected from the infrastructure or equipment (e.g., a pipeline wall) or from metal coupons positioned into the flow path or at other sites of interest to detect and characterize the biofilm and/or to evaluate metal loss and/or corrosion rate. Such factors can be assessed by the user to determine a tailored enzyme-based treatment.

The disclosed enzyme treatment methods may also be combined with other MIC-mitigation strategies.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

When a parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. The scope of the invention is not intended to be limited to the specific values and examples as recited in the specification.

### EXAMPLES

### Example 1: Controlling MIC Using the Enzymes of the Present Application in a Flow Loop System

### Stock Culture Preparation

The North Sea Sediment Enrichment (NSSE) consortium was used to evaluate corrosion remediation in a flow loop system, as described in Eibergen et al., “The Impact of Biocide Choice and Dosing Strategy on Successful MIC Management Under Anoxic and Flowing Conditions,” CORROSION 2018 (NACE International, Phoenix, Arizona, USA), NACE-2018-11295 (51318-11295-SG) (onepetro.org/NACECORR/proceedings-abstract/CORR18/All-CORR18/NACE-2018-11295/126108). The consortium included anaerobic microorganisms of the genera Desulfovibrio, Desulfocarbo, Desulfobulbus, Dethiosulfatibacter, Desulfatibacillum, Thioalbus, among other anaerobic microorganisms. To prepare an inoculum, bacteria were scraped from the metal surface in the stock culture using a sterile 5 mL serological pipette and cultured in a 125 mL serum bottle with approximately 110 mL Artificial Sea Water (ASW) media, using a process described by Widdel et al., “Gram-negative Mesophilic Sulfate-Reducing Bacteria,” in Balows et al., The Prokaryotes, 2^{nd} ed., vol. 6 (New York, NY: Springer, 1992), p. 3352; and 6-8 acid-washed sterile flow loop coupons. The culture was incubated at 30° C. for 2 weeks in anaerobic conditions, and then was sonicated for 30 s in a water bath before using the suspended microbial community as an inoculum in the flow loops. All steps were performed under anaerobic conditions.

### Metal Coupon Preparation

Coupons were acid-washed using inhibited HCl solution (by adding 50 mL concentrated HCl in 0.5 g N, N-dibutylthiourea, and then adding 50 mL dH₂O). Coupons were incubated in the solution for 1-2 minutes, and then neutralized in sodium bicarbonate solution (15 g bicarbonate dissolved in 100 ml of dH₂O) before rinsing with 100% ethanol and acetone. The same procedure was followed to clean coupons before and after experiments. Coupons were stored in acetone after cleaning.

### Flow Loop Experiments

The enzyme candidates were evaluated for MIC remediation in the flow loop system. Each flow loop was connected to a modified Robbin's device that holds C1018 carbon steel coupons with a 200-grit finish, and was recirculated at 80 min/L. The loops were pre-inoculated with the NSSE consortia for 2-3 weeks to establish biofilms under anaerobic conditions. After inoculation, the loops were treated with enzyme for a contact time of about 90 minutes or 4 hours once per week for 3 or 4 weeks. The untreated control coupons which were subjected to the inoculation were kept in the flow loops for the same total amount of time as the treated metal coupons but in the absence of any of the enzyme treatments. There were three loops run per treatment condition, and two coupons were collected and cleaned from each loop immediately post treatment for weight loss measurement according to the protocol outlined in NACE Standard RP0775-2005. A majority of the microbial composition of the biofilm was determined to be of the anaerobic Deltaproteobacteria class, based on Next-Generation Sequencing (NGS) analysis.

TABLE 1
List of representative enzymes

| Product name | Category/Type of Enzyme |
|---|---|
| Optimash DCO+ ® | Thermostable serine protease |
| Effectenz ® P100 | Thermostable and alkaline |
| | serine protease |
| PREFERENZ ™ P300 | Thermostable and alkaline |
| | serine protease |
| Optimash ® AX | Xylanase |
| Optimash ® BG | Beta-glucanase |
| Optimash ® VR | Cellulase |
| Optimash ® F200 | Cellulase, xylanase, beta-glucosidase |
| Spezyme ® HT | Alpha-amylase |

### Results

The measured weight loss was normalized by subtracting the weight loss associated with the initial 2-3 week inoculation period from the total weight loss determined over the full time period of the flow loop operation. For the untreated control metal coupons kept in the flow loop for 4 weeks following the 2-3 week inoculation period, the normalized measured weight loss ranged from 90-120 mg and the corrosion rate ranged from 45-63 mpy (1.143 mm/yr-1.60 mm/yr). For the untreated control metal coupons kept in the flow loop for 3 weeks following the 2-3 week inoculation period, the normalized weight loss ranged from 46-75 mg and the corrosion rate ranged from 33-41 mpy (0.8382 mm/yr-1.0414 mm/yr).

TABLE 2
MIC control by the protease Optimash ® DCO+

| | | | Normalized | Unnormalized | Corrosion |
|---|---|---|---|---|---|
| | | | weight loss | weight loss | rate |
| | | | reduction | reduction | reduction |
| | | | after 4 | after 4 | after 4 |
| | | | weekly | weekly | weekly |
| | | | treatments | treatments | treatments |
| | | | (% relative to | (% relative to | (% relative to |
| | Contact | | untreated | untreated | untreated |
| Concentration | time | | control) | control) | control) |
| 10 ppm | 4 | hr | 46% | 40% | 40% |
| 25 ppm | 4 | hr | 56% | 50% | 50% |
| 50 ppm | 4 | hr | 51% | 46% | 46% |
| 100 ppm | 90 | min | 59% | 46% | 46% |
| 150 ppm | 90 | min | 46% | 38% | 38% |

TABLE 3
MIC control by proteases

| | | | Normalized | Unnormalized | Corrosion |
|---|---|---|---|---|---|
| | | | weight loss | weight loss | rate |
| | | | reduction | reduction | reduction |
| | | | after 4 | after 4 | after 4 |
| | | | weekly | weekly | weekly |
| | | | treatments | treatments | treatments |
| | | | (% relative to | (% relative to | (% relative to |
| | | Contact | untreated | untreated | untreated |
| Enzyme | Concentration | time | control) | control) | control) |
| Effectenz ® | 100 ppm | 4 hr | 50% | 39% | 39% |
| P100 | | | | | |
| PREFERENZ ™ | 100 ppm | 4 hr | 50% | 45% | 45% |
| P 300 | | | | | |

TABLE 4
MIC control by carbohydrolases

| | | | Normalized | Unnormalized | Corrosion |
|---|---|---|---|---|---|
| | | | weight loss | weight loss | rate |
| | | | reduction | reduction | reduction |
| | | | after 4 | after 4 | after 4 |
| | | | weekly | weekly | weekly |
| | | | treatments | treatments | treatments |
| | | | (% relative to | (% relative to | (% relative to |
| | | Contact | untreated | untreated | untreated |
| Enzyme | Concentration | time | control) | control) | control) |
| Optimash ® AX | 100 ppm | 4 hr | 58% | 46% | 46% |
| | | | (after 4 | (after 4 | (after 4 |
| | | | weekly | weekly | weekly |
| | | | treatments) | treatments) | treatments) |
| Optimash ® BG | 100 ppm | 4 hr | 41% | 34% | 34% |
| Optimash ® VR | 100 ppm | 4 hr | 47% | 34% | 34% |
| Optimash ® F200 | 100 ppm | 4 hr | 35% | 23% | 23% |
| Spezyme ® HT | 100 ppm | 4 hr | 54% | 44% | 44% |

### Example 2 (not of the Invention)

Following the same procedure and conditions described above for Example 1, metal coupons were treated with 80 ppm of peroxidase for a contact time of 4 hours once per week for 4 weeks. No reduction in weight loss or corrosion rate was observed for the metal coupons treated with peroxidase compared to the untreated control metal coupons.

### Example 3: Treatment of Pipeline

Prior to initiating treatment, inspection and analysis of metal coupons from the targeted pipeline after at least eight weeks of exposure under anaerobic conditions indicate the presence of SRM-containing biofilms and show measurable weight loss and a corrosion rate of 5 mpy (0.127 mm/yr) or higher. An enzyme-containing composition of the present disclosure can be injected into the pipeline once-per-week for about four hours, resulting in a concentration of about 100 ppm of the enzyme in the flowing fluid during the four-hour Injection windows. After at least eight weeks of once-per-week injections, corrosion coupon measurements indicate a significant weight loss reduction and a corrosion rate reduction of at least 30%, preferably at least 40%, relative to the corrosion rate of the untreated coupon(s).

1. A method of controlling microbiologically influenced corrosion of a site of interest, comprising contacting a biofilm formed under anaerobic conditions on a metal surface of the site of interest with an effective amount of an enzyme selected from the group consisting of a protease, a carbohydrolase, a DNase and a combination thereof.

2. The method of claim 1, wherein the enzyme is selected from the group consisting of a protease, a carbohydrolase and a combination thereof.

3. The method of claim 2, wherein the enzyme is a protease selected from the group consisting of a metalloprotease, a thermostable protease, an alkaline protease and a combination thereof.

4. The method of claim 2, wherein the enzyme is a serine protease.

5. The method of claim 2, wherein the enzyme is a carbohydrolase selected from the group consisting of a glucosidase, an amylase, a xylanase and a combination thereof.

6. The method of claim 5, wherein the enzyme is a glucosidase selected from the group consisting of a cellulase, a glucanase, a beta-glucosidase and a combination thereof.

7. The method of claim 1, wherein the enzyme is selected from the group consisting of a metalloprotease, a thermostable protease, an alkaline protease, a cellulase, a xylanase, an amylase, a glucanase, a beta-glucosidase, and a combination thereof.

8. The method of claim 1, wherein the site of interest is infrastructure or equipment for refining, storing, or transporting crude or processed oil.

9. The method of claim 1, wherein the site of interest is infrastructure or equipment for processing, storing, or transporting natural gas.

10. The method of claim 1, wherein the effective amount of the enzyme is from about 10 ppm to about 500 ppm.

11. The method of claim 10, wherein the effective amount of the enzyme is from about 10 ppm to about 250 ppm.

12. The method of claim 1, wherein the biofilm comprises sulfate-reducing microorganisms.

13. The method of claim 1, wherein the effective amount of the enzyme is applied at the site of interest for a contact time, and optionally at intervals or frequencies, to provide a corrosion rate reduction of at least 20% compared to the corrosion rate of the site of interest in the absence of treatment with the enzyme.

14. A composition for controlling microbiologically influenced corrosion (MIC) in an anaerobic environment, comprising an enzyme selected from the group consisting of a protease, a carbohydrolase, a DNase and a combination thereof in an effective amount to control the MIC, wherein the effective amount is from about 1 ppm to about 500 ppm.

15. The composition of claim 14, wherein the enzyme is selected from the group consisting of a protease, a carbohydrolase and a combination thereof.

16. The composition of claim 15, wherein the enzyme is a protease selected from the group consisting of a metalloprotease, a thermostable protease, an alkaline protease and a combination thereof.

17. The composition of claim 15, wherein the enzyme is a serine protease.

18. The composition of claim 15, wherein the enzyme is a carbohydrolase selected from the group consisting of a glucosidase, an amylase, a xylanase and a combination thereof.

19. The composition of claim 18, wherein the enzyme is a glucosidase selected from the group consisting of a cellulase, a glucanase, a beta-glucosidase and a combination thereof.

20. The composition of claim 14, wherein the enzyme is selected from the group consisting of a metalloprotease, a thermostable protease, an alkaline protease, a cellulase, a xylanase, an amylase, a glucanase, a beta-glucosidase, and a combination thereof.
